(19) United States
(12) Patent Application Publication
(10) Pub. No.: US 2026/0134957 A1
(43) Pub. Date: May 14, 2026

(54) MANAGING CLINICAL TRIAL PROGRESSION USING MACHINE LEARNING-BASED DATA

(71) Applicant: Genentech, Inc., South San Francisco, CA (US)

(72) Inventors: James LU, South San Francisco, CA (US); Erick Francisco VELASQUEZ, Antioch, CA (US)

(21) Appl. No.: 19/385,930

(22) Filed: Nov. 11, 2025

Related U.S. Application Data
(63) Continuation of application No. PCT/US2024/029177, filed on May 13, 2024.
(60) Provisional application No. 63/501,991, filed on May 12, 2023.

(51) Int. Cl. G16H 10/20; G16H 50/70

(52) U.S. Cl. CPC G16H 10/20; G16H 50/70

(57) ABSTRACT

Systems and methods for managing progression of a clinical trial. Input data for a machine learning model is formed, based on longitudinal data for clinical trial cohort. The input data corresponds to input features and the cohort includes a plurality of subjects. A clinical outcome output is generated for each subject, using the machine learning model and a portion of the input data corresponding to each subject. Feature importance values are generated, based on the machine learning model generating the clinical outcome output for each subject. The feature importance values include, for each subject, a set of feature importance values for a set of input features. A ratio of interest is computed using the plurality of feature importance values. An output is generated using the ratio of interest in which the output indicates whether the cohort should proceed to a next phase of the clinical trial.

### CROSS-REFERENCE TO RELATED APPLICATIONS

This application is a continuation of International Application No. PCT/US2024/029177 filed May 13, 2024 and entitled “MANAGING CLINICAL TRIAL PROGRESSION USING MACHINE LEARNING-BASED DATA,” which claims the benefit of the priority date of U.S. Provisional Patent Application No. 63/501,991 filed May 12, 2023, entitled ‘Predicting Clinical Trial Progress Using Tumor Growth Inhibition Metrics,” each of which is incorporated herein by reference in its entirety.

### TECHNICAL FIELD

This description is generally directed towards managing the progression of a clinical trial and, more particularly, to methods and systems for managing the progression of a clinical trial based on machine learning-based data.

### BACKGROUND

Clinical trials often involve multiple phases. At each phase of clinical trial, it may be important to evaluate whether or not to proceed to a next phase of clinical trial. For example, proceeding to a next phase of clinical trial when there is a low probability of treatment response (e.g., treatment efficacy) may be more expensive and consume more resources (e.g., time resources, human resources, computing resources, etc.) than desired.

Various types of metrics may be used to assess treatment response (e.g., treatment efficacy) in patients that are part of a clinical trial. For example, tumor growth rate, tumor shrinkage rate, and time to growth/regrowth are examples of such metrics. These metrics (or parameters) may also be referred to as tumor kinetic metrics (or parameters) or tumor growth inhibition metrics (or parameters). Some currently available methodologies use tumor growth rate as a factor for determining whether to proceed to a next phase of clinical trial. However, making decisions based on tumor growth rate may be challenging as not all patients in a given cohort of the clinical trial will have tumor growth rate data. These types of methodologies may not provide desired, clinically reliable levels of predictive performance when predicting whether to proceed to Phase II from Phase I or to Phase III from either Phase II. Thus, it may be desirable to have methods and systems that recognize and consider these issues.

### SUMMARY

In one or more embodiments, a method for managing progression of a clinical trial is provided. Input data for a machine learning model is formed, based on longitudinal data for a cohort of the clinical trial, wherein the input data corresponds to a plurality of input features and wherein the cohort includes a plurality of subjects. For each subject in the plurality of subjects, a clinical outcome output is generated using the machine learning model and a portion of the input data corresponding to each subject. A plurality of feature importance values is generated, based on the machine learning model generating the clinical outcome output for each subject in the plurality of subjects, wherein the plurality of feature importance values comprises, for each subject in the plurality of subjects, a set of feature importance values for a set of selected features from the plurality of input features. A ratio of interest is computed using the plurality of feature importance values. An output is generated using the ratio of interest in which the output indicates whether to allow the cohort to proceed to a next phase of the clinical trial using the ratio of interest.

In one or more embodiments, a method for managing progression of a clinical trial is provided. For each subject in a plurality of subjects in a cohort of a clinical trial, a clinical outcome output is generated using a machine learning model and input data corresponding to a plurality of input features. A plurality of feature importance values is generated based on the machine learning model generating the clinical outcome output for each subject in the plurality of subjects, wherein the plurality of feature importance values comprises, for each subject in the plurality of subjects, a set of feature importance values for a set of selected features from the plurality of input features. A ratio of interest is computed using the plurality of feature importance values. An output is generated using the ratio of interest, wherein the output includes a positive recommendation to allow the cohort to proceed to a next phase of the clinical trial when the ratio of interest is below a decision threshold. A treatment protocol is administered to the cohort according to a clinical trial protocol associated with the next phase of the clinical trial based on the positive recommendation.

In one or more embodiments, a system comprises one or more data processors and a non-transitory computer readable storage medium containing instructions which, when executed on the one or more data processors, form input data for a machine learning model based on longitudinal data for a cohort of the clinical trial, wherein the input data corresponds to a plurality of input features and wherein the cohort includes a plurality of subjects; generate, for each subject in the plurality of subjects, a clinical outcome output using the machine learning model and a portion of the input data corresponding to each subject; generate a plurality of feature importance values based on the machine learning model generating the clinical outcome output for each subject in the plurality of subjects, wherein the plurality of feature importance values comprises, for each subject in the plurality of subjects, a set of feature importance values for a set of selected features from the plurality of input features; and compute a ratio of interest using the plurality of feature importance values; and generate an output using the ratio of interest in which the output indicates whether to allow the cohort to proceed to a next phase of the clinical trial.

In one or more embodiments, a system comprises one or more data processors and a non-transitory computer readable storage medium containing instructions which, when executed on the one or more data processors, cause the one or more data processors to perform part or all of one or more methods disclosed herein or a portion thereof.

In one or more embodiments, a computer-program product tangibly embodied in a non-transitory machine-readable storage medium, including instructions configured to cause one or more data processors to perform part or all of one or more methods disclosed herein or a portion thereof.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of an analysis system 100, in accordance with various embodiments.

FIG. 2 is a flowchart of a process for managing progression of a clinical trial using machine learning-based data in accordance with one or more embodiments.

FIG. 3 is a flowchart of a process for managing progression of a clinical trial using machine learning-based data in accordance with one or more embodiments.

FIG. 4 is a flowchart of a process for adjusting a clinical trial protocol in accordance with one or more embodiments.

FIG. 5 is a flowchart of a process for identifying a decision threshold for evaluating a ratio of interest (SEAR) in accordance with one or more embodiments.

FIG. 6 is a plot of tumor growth rate (KG) values versus SHAP values for KG in accordance with one or more embodiments.

FIG. 7 is an illustration of a plot of operating characteristic curves in accordance with one or more embodiments.

FIG. 8 is a block diagram of a computer system in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The embodiments described herein recognize and take into account that it may be desirable to have methods and systems for managing the progression of a clinical trial using machine learning-based data. For example, managing the progression of a clinical trial may include, for example, making decisions about whether to proceed from one phase of the clinical trial to another phase (e.g., a GO-decision or a NO-GO decision). Some currently available methods for making these types of decisions may not have the desired levels of accuracy and/or reliability.

In some cases, tumor kinetic data that is derived from longitudinal data (e.g., tumor size data) may be used to inform decisions about clinical trial progression. For example, tumor growth rate (KG) for the subjects in a cohort of a clinical trial may be used to inform decisions about clinical trial progression. Specifically, the geometric mean ratio GMR of tumor growth rate for a cohort may be used to inform decisions about whether that cohort should proceed to the next phase of the clinical trial. The geometric mean ratio is a formula that looks at tumor growth rate in the treatment arm versus tumor growth rate in the control arm. Typically, a treatment that is effective (e.g., relatively more effective than the control arm) results in a GMR that is less than 1. Thus, a lower GMR indicates a good treatment response (e.g., good treatment efficacy) and can be used as an indicator to allow the cohort to proceed to the next phase of clinical trial.

However, in many cases, tumor growth rate (and possibly other tumor kinetic parameters) are either not available or are non-evaluable for one or more subjects of the cohort in the clinical trial. In such situations, using the GMR for tumor growth rate to make predictions about whether to progress in a clinical trial or not may be less accurate and/or reliable than desired. In other words, the predictive performance of using the GMR for tumor growth rate may be less than desired.

Thus, the embodiments described herein provide methods and systems for managing the progression of a clinical trial using machine learning-based data. Specifically, the embodiments described herein recognize that a machine learning model that is specifically trained and configured to predict a clinical outcome output (e.g., overall survival) in a clinical trial (e.g., early phase of clinical trial) may be one measure of the treatment efficacy for the treatment arm of the clinical trial. Further, the importance of the various input features for which input data is sent into the machine learning model with respect to generating the clinical outcome output may be measured using ML-driven analysis (e.g., SHapely Additive explanations analysis or “SHAP” analysis). SHAP values may be generated for various input features even when the data for a given subject does not include data for all of the input features because the SHAP analysis can infer the importance of the features. Thus, while the input data to the machine learning model may not include tumor kinetic data (e.g., tumor growth rate data) for all subjects of the cohort of the clinical trial, there may be SHAP values for all the tumor kinetic features (e.g., tumor growth rate (KG)) for all subjects.

The embodiments described herein recognize that the log of tumor growth rate (e.g., log (KG)) is approximately equal to the SHAP values for KG (e.g., SHAP(KG). Thus, an exponential average ratio for the SHAP values of KG may be used in a manner similar to how the GMR is used for KG. This SHAP exponential average ratio (SEAR) may be compared to a threshold to determine whether to proceed to the next phase of the clinical trial. Using SEAR based on these SHAP values for KG may lead to improved predictive performance as compared to using the GMR for KG to predict whether to proceed to the next phase in a clinical trial. Further, because SHAP values are additive, the SHAP values may be combined for the purposes of the SEAR computation and used to provide even further improved performance.

The embodiments described herein may provide improved predictive performance for GO and NO-GO decisions in situations where the size of a cohort may be small and/or where tumor kinetic data is not available or non-evaluable for at least some of the subjects of the cohort. The embodiments described herein may enable more accurate clinical trial decision making, even at the time of Phase I clinical trials as compared to currently available decision-making frameworks. This improved predictive performance is an improvement to a computer system that is specifically configured for making such predictions and/or an improvement to the technology or technological field of automatically making clinical trial decisions. Still further, the embodiments described herein may lead to a conservation of computing resources, human resources, and time by ensuring the accuracy of these kinds of predictions. Additionally, the embodiments described herein provide improvements to the technology of administering treatment in clinical trials (e.g., cancer treatments).

### II. Example System for Managing Clinical Trial Progression Using Machine Learning-Based (ML-Based) Data

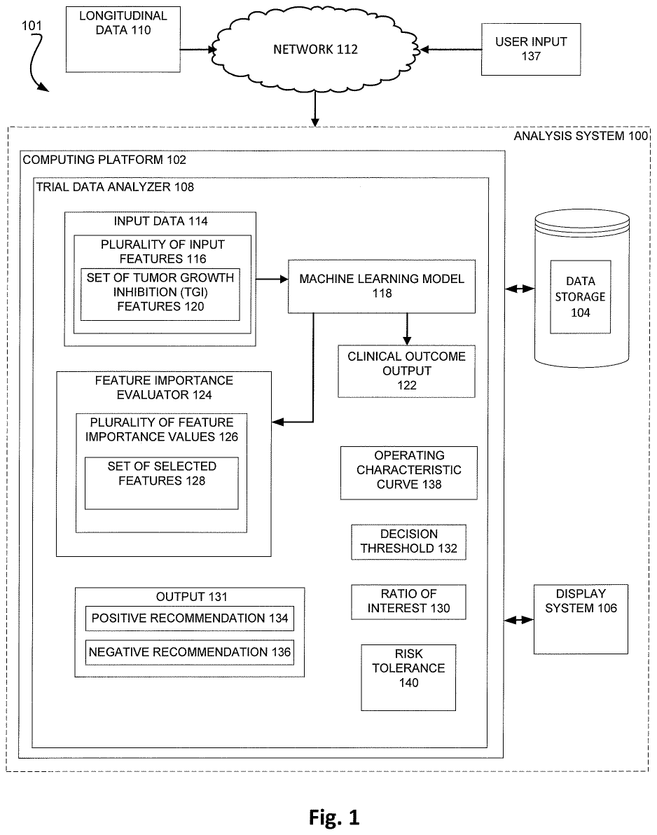

FIG. 1 is a block diagram of an analysis system 100, in accordance with various embodiments. Analysis system 100 may be used to make decisions in clinical trial settings such as, for example, without limitation, clinical trial environment 101. For example, analysis system 100 may be used to provide a machine learning based analysis of one or more phases (or stages) of a clinical trial to provide or enable decision-making about the progression of the clinical trial and/or administration of treatment as part of the clinical trial. For example, the decision-making may include decisions about whether to proceed with one or more subsequent clinical trial phases.

Analysis system 100 may be implemented using hardware, software, firmware, or a combination thereof. In one or more embodiments, analysis system 100 may include a computing platform 102, a data storage 104 (e.g., database, server, storage module, cloud storage, etc.), and a display system 106. Computing platform 102 may take various forms. In one or more embodiments, computing platform 102 includes a single computer (or computer system) or multiple computers in communication with each other. In other examples, computing platform 102 takes the form of a cloud computing platform, a mobile computing platform (e.g., laptop, a smartphone, a tablet, etc.), another processor-based device (e.g., a workstation or desktop computer) or a wearable computing device (e.g., a smartwatch), and/or a combination thereof.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Analysis system 100 includes trial data analyzer 108 that may be implemented using hardware, software, firmware, or a combination thereof. In one or more embodiments, trial data analyzer 108, which may be also referred to as a clinical trial data analyzer, is implemented using computing platform 102. Trial data analyzer 108 may receive longitudinal data 110 for processing.

In one or more embodiments, trial data analyzer 108 may receive longitudinal data 110 over network 112. Network 112 may be implemented using a single network or multiple networks in combination. Network 112 may be implemented using any number of wired communications links, wireless communications links, optical communications links, or combination thereof. For example, in various embodiments, network 112 may include the Internet or one or more intranets, landline networks, wireless networks, and/or other appropriate types of networks. In another example, the network 112 may comprise a wireless telecommunications network (e.g., cellular phone network) adapted to communicate with other communication networks, such as the Internet. In some cases, network 112 includes at least one of a local area network (LAN), a virtual local area network (VLAN), a wide area network (WAN), a public land mobile network (PLMN), the Internet, or another type of network.

Longitudinal data 110 may be data associated with a clinical trial. For example, longitudinal data 110 may include data associated with one or more phases of a clinical trial. In one or more embodiments, longitudinal data 110 includes data for a plurality of subjects in a cohort of the clinical trial. The cohort may be, for example, subjects within a particular treatment arm of the clinical trial. Longitudinal data 110 may include, for example, data relating to a set of variables that corresponds to a period of time, which may also be referred to as an initial period of time. This initial period of time may be, for example, one week, one month, two months, 3 months, 6 months, or some other period of time. In various embodiments, the longitudinal data corresponds to an initial period of time that includes a portion of time after the administration of a treatment, as well as at least one point in time prior to treatment. As one example, the longitudinal data may include a baseline value for a tumor-related variable (e.g., collected pre-treatment) and values for the tumor-related variable for a portion of time (e.g., 4 weeks) post-treatment. The set of variables may include only a single tumor-related variable (e.g., tumor size) or may include multiple tumor-related variables, including tumor size. Tumor size may include, for example, tumor volume.

Trial data analyzer 108 may use longitudinal data 110 and optionally, other types of data, to form input data 114 corresponding to a plurality of input features 116 for a machine learning model 118. A feature may also be referred to as a parameter.

For example, trial data analyzer 108 may use longitudinal data 110 and more specifically, tumor kinetic data derived from longitudinal data 110, as well as baseline data to form input data 114. The baseline data may include values for a plurality of baseline features (or parameters or covariates), which may include, for example, at least one of age, gender, baseline C-reactive protein (CRP), baseline albumin, or one or more other types of covariates.

The tumor kinetic data derived from the longitudinal data may include values for a plurality of tumor kinetic features (or parameters). These tumor kinetic features may include, but are not limited to, tumor growth inhibition (TGI) features. In one or more embodiments, the tumor kinetic data includes values, computed or otherwise derived from the longitudinal data, for time to tumor regrowth (TTG), tumor growth rate (KG), tumor shrinkage rate (KS), or a combination thereof. Thus, input features 116 may include set of tumor growth inhibit (TGI) features 120, which may include one or more of time to tumor regrowth (TTG), tumor growth rate (KG), tumor shrinkage rate (KS).

Thus, the plurality of input features 116 may include, for example, one or more features selected from the group consisting of tumor growth rate (KG), C-reactive protein level (CRP), time to tumor growth/regrowth (TTG), baseline neutrophil/lymphocyte ratio (BNLR), baseline Eastern Cooperative Oncology Group score (ECOG) score, liver metastasis level (LIVER), tumor shrinkage rate (KS), hemoglobin level (HGB), time since initial diagnosis (TSD), total protein (TPRO), albumin level (ALBU), and number of metastatic sites at enrollment (METSITES). The time since initial diagnosis, when in years, may be referred to as years since initial diagnosis (YSD).

In one or more embodiments, the portion of input data 114 corresponding to a given subject in the cohort in the clinical trial may not include values for one or more TGI features included in plurality of input features 116. For example, in some cases, not all subjects in the cohort may have a tumor growth rate (KG). These subjects for which one or more TGI features are excluded (or missing, omitted, etc.) may be referred to as TGI-non evaluable subjects, in some cases.

Machine learning model 118 processes input data 114 to generate clinical outcome output 122 for each subject of the cohort in the clinical trial. Clinical outcome output 122 may include, for example, an overall survival, a hazard ratio computed for overall survival, both, some other type of survival metric, or a combination thereof. Machine learning model 118 may include, for example, a pan-indication model (or pan gradient boosting model. This pan-indication gradient boosting model may include, for example, a gradient boosting decision tree-based ensemble machine learning algorithm (e.g., XGBoost).

In addition to machine learning model 118, trial data analyzer 108 may include feature importance evaluator 124 that evaluates the importance of the input features 116 based on machine learning model 118 generating clinical outcome output 122 to thereby generate plurality of feature importance values 126. Feature importance values 126 include, for each subject in the cohort, a set of feature importance values for a set of selected features 128 that have been selected from input features 116. For example, set of selected features 128 may include one or more features from set of TGI features 120 (e.g., tumor growth rate (KG), tumor shrink rate (KS), or time to tumor growth/regrowth (TTG)). In one or more embodiments, set of selected features 128 includes KG. In other embodiments, set of selected features 128 includes KG, KS, and TTG, or some other combination of KG, KS, and TTG.

Plurality of feature importance values 126 may include, for any given subject, a feature importance value for each selected feature in set of selected features 128, regardless of whether input data 114 included a value for that same feature for the given subject. Feature importance evaluator 124 may thus be capable of inferring the importance of certain features, regardless of whether a value for that feature was input into machine learning model 118.

Feature importance values 126 may include, for example, values derived using SHapley Additive explanations analysis (“SHAP analysis”). These values may thus be referred to as SHAP values. Feature importance values 126 may, for example, include SHAP values for tumor growth rate (KG), tumor shrink rate (KS), time to tumor growth/regrowth (TTG), or a combination thereof.

Trial data analyzer 108 may use feature importance values 126 to compute an effect size, which may be, for example, ratio of interest 130. Ratio of interest 130 may be, for example, an exponential average ratio. For example, when feature importance values 126 take the form of SHAP values, ratio of interest 130 may be referred to as a SHAP exponential average ratio or SEAR. SEAR(KG), which may be the SHAP exponential average ratio for SHAP values for tumor growth rate (KG), may be computed as follows:

$$SEAR ( {KG}_{t} , {KG}_{c} ) = \frac{exp ( \frac{1}{n} ( {\sum }_{i = 1}^{n} SHAP ( {KG}_{ti} ) )}{exp ( \frac{1}{n} ( {\sum }_{i = 1}^{n} SHAP ( {KG}_{ci} ) )} , \qquad ( 1 )$$

where t refers to the treatment arm and c refers to the control arm.

Based on the additive property of SHAP values where:

$$SHAP ( TGI ) = SHAP ( K G ) + SHAP ( TTG ) + SHAP ( KS ) , \qquad ( 2 )$$

and based on the generalization that SHAP (KG) may be generalized to SHAP (TGI),

$$SEAR ( {TGI}_{t} , {TGI}_{c} ) = \frac{exp ( \frac{1}{n} ( {\sum }_{i = 1}^{n} SHAP ( {TGI}_{ti} ) )}{exp ( \frac{1}{n} ( {\sum }_{i = 1}^{n} SHAP ( {TGI}_{ci} ) )} . \qquad ( 3 )$$

Ratio of interest 130 is expected to be less than one and smaller when the treatment arm is effective as compared to the control arm of the clinical trial. For example, the more effective the treatment arm, the smaller ratio of interest 130 may be.

Ratio of interest 130 may be used to generate output 131. For example, ratio of interest 130 may be compared to decision threshold 132 to determine whether ratio of interest 130 is above, at, or below decision threshold 132. This comparison may be used to generate output 131. For example, when ratio of interest 130 (e.g., SEAR) is below (or at or below) the decision threshold 132, output 131 may include a positive recommendation 134 that recommends proceeding to the next phase of the clinical trial. On the other hand, when ratio of interest 130 is above (or at or above) the decision threshold 132, output 131 may include a negative recommendation 136 that does not recommend proceeding to the next phase of the clinical trial. The positive recommendation 134 may be referred to as a GO-decision, while the negative recommendation 136 may be referred to as a NO-GO-decision.

Decision threshold 132 may be, for example, 0.8, 0.9, 0.95, or some other value selected between, for example, 0.6 and 0.98. A lower decision threshold 132 may be selected to reduce the possibility that a false positive recommendation (e.g., an incorrect GO decision) is made. In other words, a lower decision threshold 132 may be selected when there is less tolerance for risk. A higher decision threshold 132 may be selected when there is a greater tolerance for risk such that the possibility that a false positive recommendation (E.g., an incorrect GO decision) is made is greater.

Decision threshold 132 may be determined in different ways. For example, decision threshold 132 may be received in user input 137 that is received over network 112. In some cases, decision threshold 132 may be computed or otherwise determined based on operating characteristic curve 138. Operating characteristic curve 138 may also be referred to as a receiver operating characteristic (ROC) curve. In one or more embodiments, trial data analyzer 108 may build operating characteristic curve 138 as a plot the probability of a true positive recommendation versus a false positive recommendation using historical clinical trial data. A false positive recommendation may be one in which a recommendation was made to proceed to the next phase of the clinical trial where the treatment would not be as effective as desired relative to the control arm. For example, for every possible value for decision threshold 132, operating characteristic curve 138 may include a probability for a false positive recommendation (e.g., incorrect GO decision) and the probability of a true positive recommendation (e.g., a correct GO decision).

In one or more embodiments, trial data analyzer 108 may identify risk tolerance 140 (e.g., via user input 137). Risk tolerance 140 may be, for example, the maximum probability that is to be allowed for a false positive recommendation. For example, when the risk tolerance 140 is 5%, this may mean that the probability of a false positive recommendation should be limited to no more than 5%.

Trial data analyzer 108 may use operating characteristic curve 138 and risk tolerance 140 to identify decision threshold 132 that is then used to evaluate ratio of interest 130 and generate output 131. As explained above, when ratio of interest 130 is above decision threshold 132, a negative recommendation 136 is included in output 131. When ratio of interest 130 is below decision threshold 132, a positive recommendation 134 is included in output 131.

Output 131 may include other information as well. For example, output 131 may include clinical outcome output 122, feature importance values 126 (or a portion thereof), and/or other types of information. In some cases, output 131 may include instructions identifying the protocol for the next phase of the clinical trial.

If output 131 includes positive recommendation 134, then a treatment protocol may be administered to the cohort in accordance with the next phase of the clinical trial (e.g., in accordance with a protocol associated with the next phase of the clinical trial). The treatment may include, for example, one or more cancer therapies. For example, the treatment may include at least one of an immunotherapy, a targeted therapy, a radiation therapy, or a chemotherapy.

If output 131 includes negative recommendation 136, then further analyses may be performed to determine whether to proceed with a subset of the cohort. For example, ratios of interest (SEARs) may be computed for different subsets of the cohort in order to determine whether a subset of the cohort may be better suited to (e.g., may experience a better treatment response) proceed to the next phase of the clinical trial.

In this manner, the systems and methods described herein enable automatically evaluating treatment efficacy or treatment response in a clinical trial in order to determine whether a given cohort should proceed to the next phase of a clinical trial and automatically generating a corresponding recommendation. The embodiments described herein with respect to FIG. 1 that use SEAR may lead to improved predictive performance as compared to using the GMR for KG to predict whether to proceed to the next phase in a clinical trial. Further, because SHAP values are additive, the SHAP values may be combined for the purposes of the SEAR computation and used to provide even further improved performance. Still further, the embodiments described herein may provide improved predictive performance for GO and NO-GO decisions in situations where the size of a cohort may be small and/or where tumor kinetic data (e.g., tumor growth data) is not available or non-evaluable for at least some of the subjects of the cohort.

The embodiments described herein may enable more accurate clinical trial decision making, even at the time of Phase I clinical trials as compared to currently available decision-making frameworks. This improved predictive performance may be an improvement to a computer system that is specifically configured for making such predictions and/or an improvement to the technology or technological field of automatically making clinical trial decisions. Still further, the embodiments described herein may lead to a conservation of computing resources, human resources, and time by ensuring the accuracy of these kinds of predictions. Additionally, the embodiments described herein provide improvements to the technology of administering treatment in clinical trials (e.g., cancer treatments).

### III. Exemplary Methodologies for Managing Clinical Trial Progression Using Machine Learning-Based (ML-Based) Data

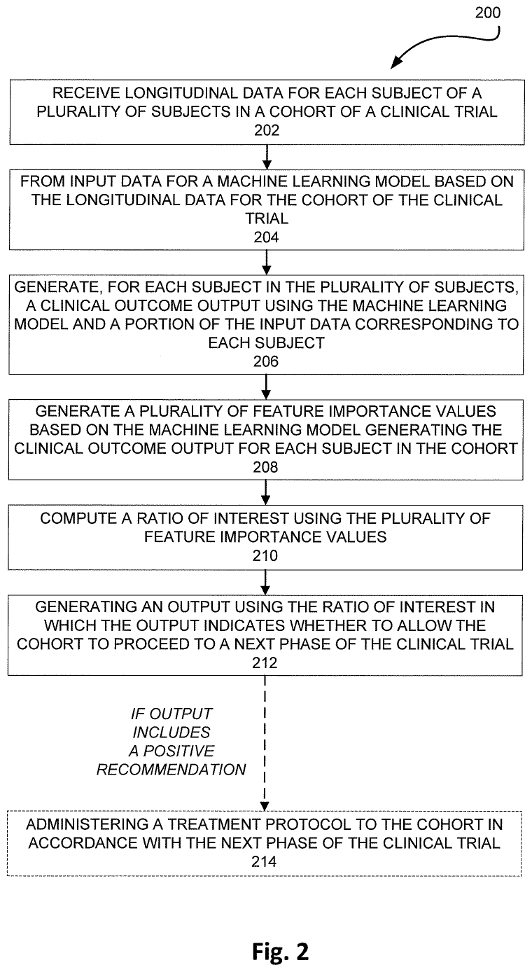

FIG. 2 is a flowchart of a process 200 for managing progression of a clinical trial using machine learning-based data in accordance with various embodiments. Process 200 may be implemented using analysis system 100 described in FIG. 1. In one or more embodiments, at least some of the steps of the process 200 may be performed by the processors of a computer or a server implemented as part of analysis system 100. It is understood that additional steps may be performed before, during, or after the steps of process 200 discussed below. In addition, in some embodiments, one or more of the steps may also be omitted or performed in different orders.

Process 200 may optionally include step 202, which includes receiving longitudinal data for each subject of a plurality of subjects in a cohort of a clinical trial. The cohort of subjects may belong to one particular arm (e.g., treatment arm) of the clinical trial. The longitudinal data may be one example of an implementation for longitudinal data 110 in FIG. 1. The longitudinal data may correspond to a first phase of a clinical trial, a second phase of a clinical trial, or both the first phase and second phase of the clinical trial. For example, the longitudinal data may include measurement data for tumor volume over time.

The longitudinal data may be data for a set of set of variables that corresponds to a period of time (e.g., initial period of time). This period of time may be, for example, one week, one month, two months, 3 months, 6 months, or some other period of time. In various embodiments, the longitudinal data corresponds to a period of time that includes a portion of time after the administration of a treatment, as well as at least one point in time prior to treatment. As one example, the longitudinal data may include a baseline value for a tumor-related variable (e.g., collected pre-treatment) and values for the tumor-related variable for a portion of time (e.g., 4 weeks) post-treatment. The set of variables may include only a single tumor-related variable (e.g., tumor size) or may include multiple tumor-related variables, including tumor size. Tumor size may include, for example, tumor volume, tumor diameter, and/or one or more other types of size-related parameters.

Step 204 includes forming input data for a machine learning model based on the longitudinal data for the cohort of the clinical trial. Step 204 may include, for example, forming the input data using baseline data and tumor kinetic data derived from the longitudinal data.

With respect to step 204, the input data, which may be one example of an implementation for input data 114 in FIG. 1, corresponds to (e.g., is generated for) a plurality of input features, which may be one example of an implementation for input features 116 in FIG. 1. The baseline data may include values for one or more baseline features (or parameters or covariates), which may include, for example, at least one of age, gender, baseline C-reactive protein (CRP), baseline albumin, or one or more other types of covariates.

The tumor kinetic data derived from the longitudinal data may include values for a plurality of tumor kinetic parameters. These tumor kinetic parameters may include, but are not limited to, tumor growth inhibition (TGI) metrics. In one or more embodiments, the tumor kinetic data includes values computed or otherwise derived from the longitudinal data for time to tumor growth/regrowth (TTG), tumor growth rate (KG), tumor shrinkage rate (KS), or a combination thereof.

In one or more embodiments, for each subject of the cohort, the input data may include one or more features selected from the group consisting of tumor growth rate (KG), C-reactive protein level (CRP), time to tumor growth/regrowth (TTG), baseline neutrophil/lymphocyte ratio (BNLR), baseline Eastern Cooperative Oncology Group score (ECOG) score, liver metastasis level (LIVER), tumor shrinkage rate (KS), hemoglobin level (HGB), time since initial diagnosis (TSD), total protein (TPRO), albumin level (ALBU), and number of metastatic sites at enrollment (METSITES). The time since initial diagnosis, when in years, may be referred to as years since initial diagnosis (YSD).

In one or more embodiments, the input data includes the same data for each subject in the cohort. In other embodiments, some subjects may not have values for certain input features. For example, the input data may include tumor growth rate (KG) values for some subjects but not for other subjects. In other words, the tumor growth rate may be excluded (e.g., missing, omitted, etc.) for certain subjects of the cohort.

Step 206 includes generating, for each subject in the plurality of subjects, a clinical outcome output using the machine learning model and a portion of the input data corresponding to each subject. The machine learning model may include, for example, a pan-indication model. The pan-indication model may include, for example, without limitation, a gradient boosting decision tree-based ensemble machine learning algorithm.

The pan-indication model may be one that has been trained using data for a selected population to process the input data and generate the clinical outcome output. The selected population may include multiple patient/subject populations across at least one clinical trial across at least one of a plurality of tumor types or a plurality of treatment types. In some embodiments, the selected population may include multiple patient/subject populations across a plurality of tumor types, a plurality of treatment types, or both.

The clinical outcome output may be one example of an implementation for clinical outcome output 122 in FIG. 1. The clinical outcome output generated in step 206 may be an indication of clinical outcome (e.g., overall survival). In other embodiments, the clinical outcome output is further processed and then used to make a prediction of the clinical outcome. In one or more embodiments, the clinical outcome output may include, but is not limited to, a hazard ratio, a hazard ratio for overall survival, a metric for overall survival, some other type of survival metric, or a combination thereof. In various embodiments, the clinical outcome predicted in step 206 is used to generate one or more hazard ratios of the treatment arm with respect to the control arm of the clinical trial. For example, the clinical outcome output generated by the pan-indication model may be a score that indicates overall survival. This score may then be used to generate a hazard ratio for overall survival.

Step 208 includes generating a plurality of feature importance values based on the machine learning model generating the clinical outcome output for each subject in the cohort. The feature importance values may be one example of an implementation for feature importance values 126 in FIG. 1. In one or more embodiments, the feature importance values include Shapely additive explanations (SHAP) values.

The feature importance values may include, for example, for each subject in the cohort, a set of feature importance values for a set of selected features from the input features. For example, for a given subject, the set of selected features may be one or more of the TGI features. For example, the set of selected features may include tumor growth rate (KG), tumor shrinkage rate (KS), time for tumor growth (TTG), or a combination thereof. In these examples, the set of feature importance values generated is generated for the same set of selected features for each subject, regardless of whether that particular input feature was associated with a value in the input data fed into the machine learning model.

For example, the portion of input data corresponding to a given subject may include a value for KS and a value for TTG but may not include a value for KG. Regardless, step 208 may include generating a feature importance value for each of KG, KS, and TTG.

Step 210 includes computing a ratio of interest using the plurality of feature importance values. The ratio of interest may be, for example, an exponential average ratio parameter. For example, when the feature importance values generated in step 208 are SHAP values, the ratio of interest may be referred to as SHAP exponential average ratio (SEAR) parameter.

Step 212 may include generating an output using the ratio of interest in which the output indicates whether to allow the cohort to proceed to a next phase of the clinical trial. Step 212 may be performed using a decision threshold. This decision threshold may be one example of an implementation for decision threshold 132 in FIG. 1. The decision threshold may be provided via, for example, user input, or may be automatically computed by the system based on a determined risk tolerance. The risk tolerance may be provided by, for example, without limitation, user input. Step 212 may be performed by comparing the ratio of interest to the decision threshold to determine whether the ratio of interest is above or below the decision threshold.

In one or more embodiments, when the ratio of interest is below the decision threshold (or at or below the decision threshold), the output generated in step 212 includes a positive recommendation that recommends proceeding to the next phase of the clinical trial. Conversely, when the ratio of interest is above the decision threshold (or at or above the decision threshold), the output generated ins step 212 includes a negative recommendation that recommends not proceeding to the next phase of the clinical trial. The positive recommendation may be referred to as a GO-decision, and the negative recommendation may be referred to as a NO-GO-decision in one or more embodiments.

Process 200 may optionally include step 214. For example, in some cases, if the output generated in step 212 includes a positive recommendation, then step 214 may be performed. Step 214 includes administering a treatment protocol to the cohort in accordance with the next phase of the clinical trial. For example, the treatment protocol may include one or more therapies (e.g., cancer therapies) to be administered to the subject according to a protocol associated with the next phase of the clinical trial. The treatment protocol may include administering, for example, without limitation, at least one of an immunotherapy (e.g., an immune checkpoint inhibitor, a monoclonal antibody therapy), a targeted therapy (e.g., a targeted cancer therapy, an anti-VEGF therapy), a radiation therapy, or a chemotherapy (e.g., an alkylating agent, mitotic inhibitor, a taxane, an antineoplastic). As one illustrative example, the treatment protocol may include administering atezolizumab, bevacizumab, paclitaxel, carboplatin, or a combination thereof.

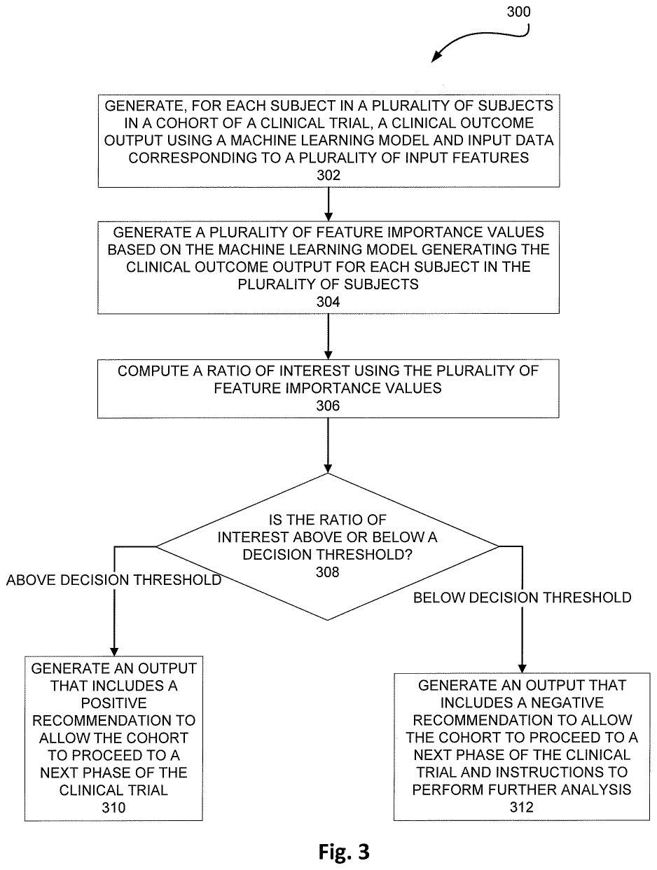

FIG. 3 is a flowchart of a process 300 for managing progression of a clinical trial using machine learning-based data in accordance with various embodiments. Process 300 may be implemented using analysis system 100 described in FIG. 1. In one or more embodiments, at least some of the steps of the process 300 may be performed by the processors of a computer or a server implemented as part of analysis system 100. It is understood that additional steps may be performed before, during, or after the steps of process 300 discussed below. In addition, in some embodiments, one or more of the steps may also be omitted or performed in different orders.

Step 302 includes generating, for each subject in a plurality of subjects in a cohort of a clinical trial, a clinical outcome output using a machine learning model and input data corresponding to a plurality of input features. The input data may be, for example, input data 114 described with respect to FIG. 1. The input features may include, for example, input features 116 in FIG. 1.

Step 304 includes generating a plurality of feature importance values based on the machine learning model generating the clinical outcome output for each subject in the plurality of subjects. The feature importance values may include, for each subject in the plurality of subjects, a set of feature importance values for a set of selected features selected from the input features. The set of selected features may include, for example, at least one of tumor growth rate (KG), tumor shrinkage rate (KS), or time for tumor growth (TTG). The feature importance values, which may be, for example, feature importance values 126 in FIG. 1, may take the form of SHAP values.

Step 306 includes computing a ratio of interest using the plurality of feature importance values. The ratio of interest may be, for example, a measure of effect size. The ratio of interest may be, for example, ratio of interest 130 in FIG. 1. The ratio of interest may be, for example, an exponential average ratio. The ratio of interest may be, for example, a SHAP exponential average ratio (SEAR). The SEAR may use the SHAP values for KG, KS, and/or TTG. For example, the SHAP values for KG, KS, and TTG may be added to form SHAP (TGI) values for which the SEAR may be computed.

Step 308 includes determining whether the ratio of interest is above or below a decision threshold. The decision threshold may be, for example, decision threshold 132 in FIG. 1.

If the ratio of interest is below the decision threshold, step 310 is performed, which includes generating an output that includes a positive recommendation to allow the cohort to proceed to a next phase of the clinical trial. Optionally, the process 300 may further include a step for administering a treatment protocol to the cohort according to a clinical trial protocol associated with the next phase of the clinical trial based on the positive recommendation.

With reference again to step 308, if the ratio of interest is above the decision threshold, step 312 is performed, which includes generating an output that includes a negative recommendation to allow the cohort to proceed to a next phase of the clinical trial and instructions to perform further analysis.

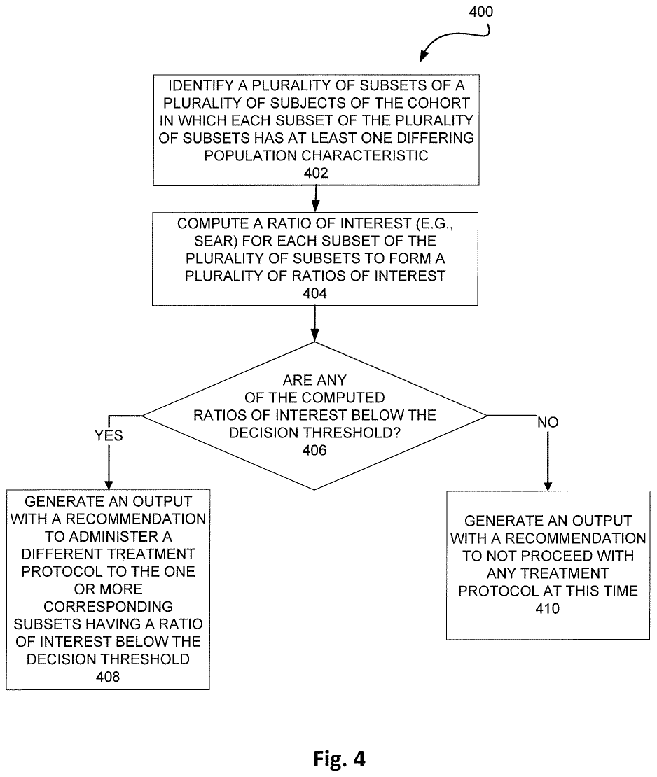

FIG. 4 is a flowchart of a process 400 for adjusting a clinical trial protocol in accordance with one or more embodiments. Process 400 may be implemented using analysis system 100 described in FIG. 1. In one or more embodiments, at least some of the steps of the process 400 may be performed by the processors of a computer or a server implemented as part of analysis system 100. It is understood that additional steps may be performed before, during, or after the steps of process 400 discussed below. In addition, in some embodiments, one or more of the steps may also be omitted or performed in different orders. Process 400 may be performed, for example, in response to the output generated in step 312 in FIG. 3.

Step 402 includes identifying a plurality of subsets of a plurality of subjects of the cohort in which each subset of the plurality of subsets has at least one differing population characteristic. Examples of population characteristics may include, but are not limited to, age, sex, gender, disease severity, and/or one or more other demographic and/or clinical factors. In one or more embodiments, the cohort in step 402 may be the same cohort described with respect to FIG. 3.

Step 404 includes computing a ratio of interest (e.g., SEAR) for each subset of the plurality of subsets to form a plurality of ratios of interest. These ratios of interest may be computed based on the feature importance values generated via process 300 in FIG. 3.

Step 406 includes determining whether any of the computed ratios of interest are below the decision threshold. This decision threshold (e.g., decision threshold 132 in FIG. 1) may be the same decision threshold described with respect to process 300 in FIG. 3.

If any of the computed ratios of interest are below the decision threshold, step 408 may be performed, which includes generating an output with a recommendation to administer a different treatment protocol to the one or more corresponding subsets having a ratio of interest below the decision threshold. Process 400 may optionally further include a step for administering the different treatment protocol.

With reference again to step 406, if none of the computed ratios of interest are below the decision threshold, step 410 may be performed, which includes generating an output with a recommendation to not proceed with any treatment protocol at this time. The ratio of interests described herein provide a good measure for the efficacy of the treatment arm with respect to the control arm in a clinical trial. Accordingly, decisions may be reliably made with respect to GO and NO-GO decisions given the desired level of risk tolerance.

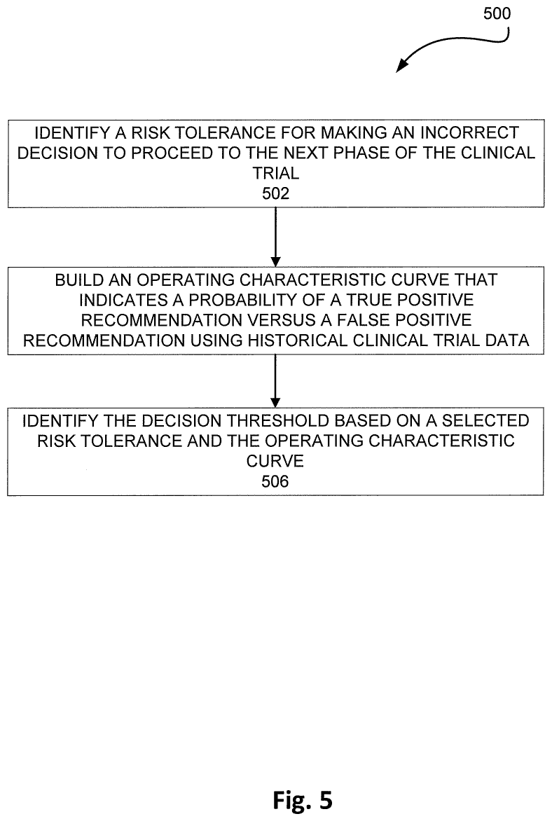

FIG. 5 is a flowchart of a process 500 for identifying a decision threshold for evaluating a ratio of interest (SEAR) in accordance with one or more embodiments. Process 500 may be implemented using analysis system 100 described in FIG. 1. In one or more embodiments, at least some of the steps of the process 500 may be performed by the processors of a computer or a server implemented as part of analysis system 100. It is understood that additional steps may be performed before, during, or after the steps of process 500 discussed below. In addition, in some embodiments, one or more of the steps may also be omitted or performed in different orders. Process 500 may be performed, for example, to determine the decision thresholds described with respect to process 300 in FIG. 3 and in process 400 in FIG. 4.

Step 502 includes identifying a risk tolerance for making an incorrect decision to proceed to the next phase of the clinical trial.

Step 504 includes building an operating characteristic curve that indicates a probability of a true positive recommendation versus a false positive recommendation using historical clinical trial data.

Step 506 includes identifying the decision threshold based on a selected risk tolerance and the operating characteristic curve.

In this manner, the methods described herein with respect to FIGS. 2-5 enable automatically evaluating treatment efficacy or treatment response in a clinical trial in order to determine whether a given cohort should proceed to the next phase of a clinical trial and automatically generating a corresponding recommendation. The embodiments described herein with respect to FIG. 1 that use SEAR may lead to improved predictive performance as compared to using the GMR for KG to predict whether to proceed to the next phase in a clinical trial. Further, because SHAP values are additive, the SHAP values may be combined for the purposes of the SEAR computation and used to provide even further improved performance. Still further, the embodiments described herein may provide improved predictive performance for GO and NO-GO decisions in situations where the size of a cohort may be small and/or where tumor kinetic data (e.g., tumor growth data) is not available or non-evaluable for at least some of the subjects of the cohort.

The embodiments described herein may enable more accurate clinical trial decision making, even at the time of Phase I clinical trials as compared to currently available decision-making frameworks. This improved predictive performance may be an improvement to a computer system that is specifically configured for making such predictions and/or an improvement to the technology or technological field of automatically making clinical trial decisions. Still further, the embodiments described herein may lead to a conservation of computing resources, human resources, and time by ensuring the accuracy of these kinds of predictions. Additionally, the embodiments described herein provide improvements to the technology of administering treatment in clinical trials (e.g., cancer treatments).

### IV. Example Experimental Study

### IVA. Example Methodology

In one example study for evaluating clinical trial decision-making frameworks, data from Impower150, a randomized Phase III study of first-line therapy in 1,202 patients with non-small cell lung cancer, was sampled 500 times to create simulations of 40 patients/arm with 6 to 24 weeks follow-up. Correct and incorrect GO decisions (e.g., decisions to proceed to Phase III) were evaluated by first comparing sampling from a first arm (e.g., atezolizumab plus bevacizumab plus chemotherapy (ABCP)) versus a sampling from a second arm that was different (e.g., bevacizumab plus chemotherapy (BCP)) and then comparing a sampling from the second arm (BCP) versus another sampling from the second arm (BCP) respectively.

Tumor kinetic data (e.g., values for tumor growth inhibition (TGI) features) were derived from modeling the sum of the longest diameters of tumor lesions over time. Specifically, KG, KS, and TTG were derived. A pan-indication machine learning model (e.g. machine learning model 118 in Figure) was trained using the TGI features and selected baseline covariate data compiled across 9 atezolizumab containing trials (6,128 patients) with the predictive target being overall survival.

Using SHapley Additive explanations (“SHAP analysis”), the contribution of TGI metrics towards predicting overall survival across patients was evaluated, ignoring the effects of covariates by setting them to their median values. Specifically, a SHAP value was derived for each TGI metric (e.g., SHAP(KG) derived for KG).

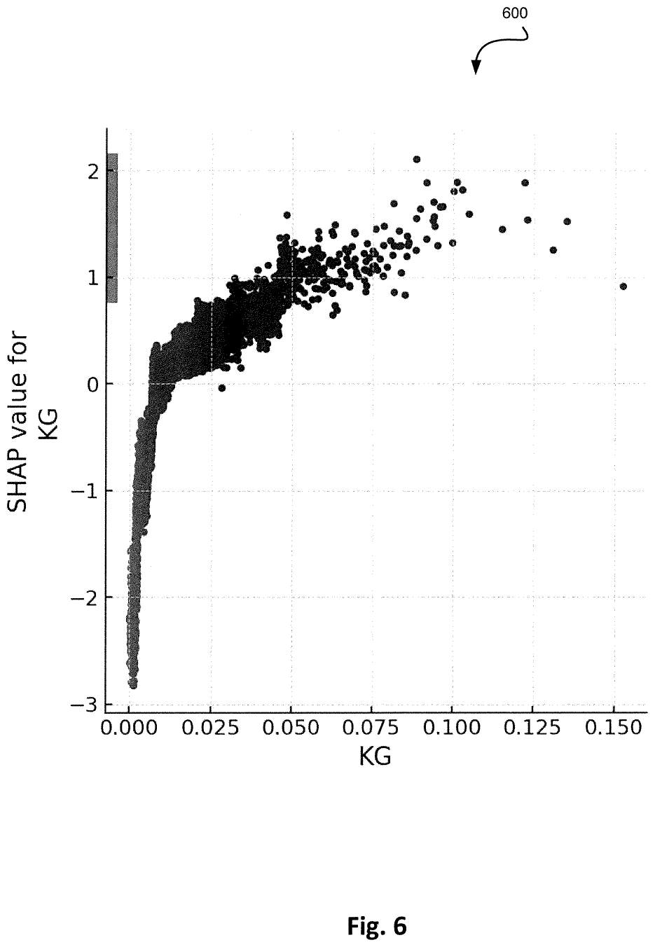

FIG. 6 is a plot 600 of tumor growth rate (KG) values versus SHAP values for tumor growth rate (KG) in accordance with one or more embodiments. As shown in plot 600, higher values of tumor growth rate (KG) may generally correspond to SHAP values that indicate a greater importance to the overall clinical outcome output generated by the pan-indication machine learning model.

Using the ansatz of a logarithmic dependence between KG and SHAP(KG), the SHAP exponential average ratio for KG was then computed as SEAR(KG). The additive property of SHAP values was used to derive a combined SEAR(TGI) using the sum of the SHAP values for KG, KS, and TTG. The performance curves (e.g., operating characteristic curves) for correct predictions and incorrect predictions of GO and NO-GO decisions were generated by scanning through values of different effect size ratio thresholds (e.g., varying values for decision threshold 132 in FIG. 1).

### IVB. Example Results

The pan-indication model using three TGI metrics (e.g., KG, KS, and TTG) to predict GO and NO-GO decisions for clinical trial purposes demonstrated improvement over previously used frameworks that were based on the Geometric Mean Ratio (GMR) of tumor growth rate (KG). Using SEAR(TGI) showed better improvement as compared to using SEAR(KG) alone. Further, with SEAR(TGI), SHAP values can also be computed for TGI non-evaluable patients, which may not be currently possible with the existing GMR framework.

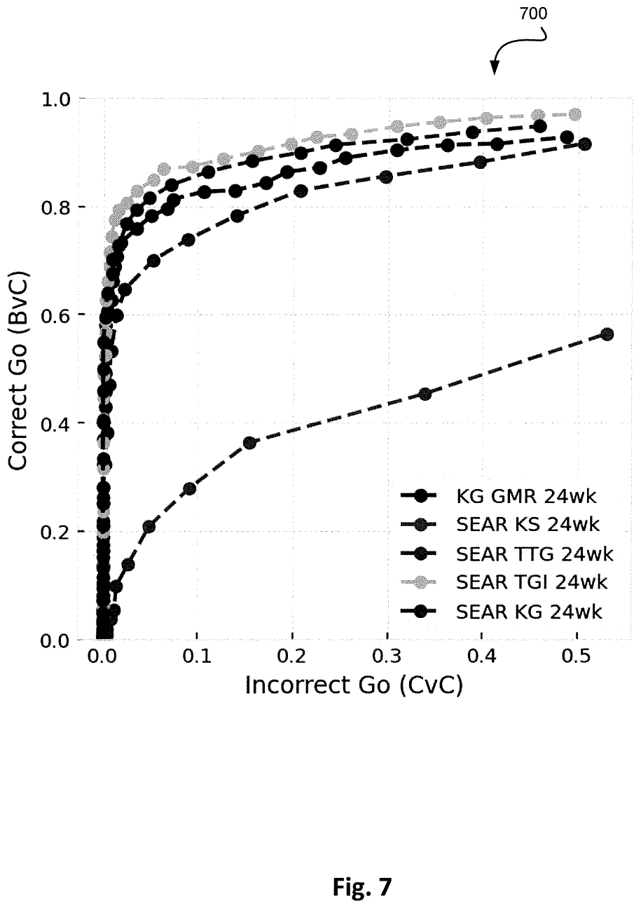

FIG. 7 is an illustration of a plot 700 of various operating characteristic curves in accordance with one or more embodiments. Plot 700 shows five operating characteristic curves based on using various types of effect sizes (e.g., geometric mean ratio (GMR) and SHAP exponential average ratio (SEAR)).

A first operating characteristic curve shows performance based on using GMR computed based on tumor growth rate (KG) values—GMR(KG)—for the 24-week follow-up visit. A second operating characteristic curve shows performance based on using SEAR computed based on SHAP(KG) values—SEAR(KG)—for the 24-week follow up visit. A third operating characteristic curve shows performance based on using SEAR computed based on SHAP(KS) values—SEAR(KS)—for the 24-week follow up visit. A fourth operating characteristic curve shows performance based on using SEAR computed based on SHAP (TTG) values—SEAR(TTG)—for the 24-week follow up visit. A fifth operating characteristic curve shows performance based on using SEAR for the combined SHAP (TGI) values—SEAR(TGI)—for the 24-week follow up visit. For SEAR(TGI), the SHAP values for KG, KS, and TTG are combined. As shown in 600, the operating characteristic curve for SEAR(TGI) shows improved overall performance as compared to the other four operating characteristic curves.

For GMR(KG), a risk tolerance of 5% (e.g., about 5% or less probability of predicting an incorrect GO decision) corresponds to about a 78% probability of predicting a correct GO decision and a decision threshold (e.g., for use in evaluating GMR) of about 0.85. For SEAR(KG), a risk tolerance of 5% (e.g., about 5% or less probability of predicting an incorrect GO decision) corresponds to about an 82% probability of predicting a correct GO decision and a decision threshold (e.g., for use in evaluating SEAR) of about 0.91. For SEAR(KS), a risk tolerance of 5% (e.g., about 5% or less probability of predicting an incorrect GO decision) corresponds to about an 21% probability of predicting a correct GO decision and a decision threshold (e.g., for use in evaluating SEAR) of about 0.95. For SEAR(TTG), a risk tolerance of 5% (e.g., about 5% or less probability of predicting an incorrect GO decision) corresponds to about an 69% probability of predicting a correct GO decision and a decision threshold (e.g., for use in evaluating SEAR) of about 0.92. For SEAR(TGI), a risk tolerance of 5% (e.g., about 5% or less probability of predicting an incorrect GO decision) corresponds to about an 85% probability of predicting a correct GO decision and a decision threshold (e.g., for use in evaluating SEAR) of about 0.85.

### V. Example Computer Implemented System

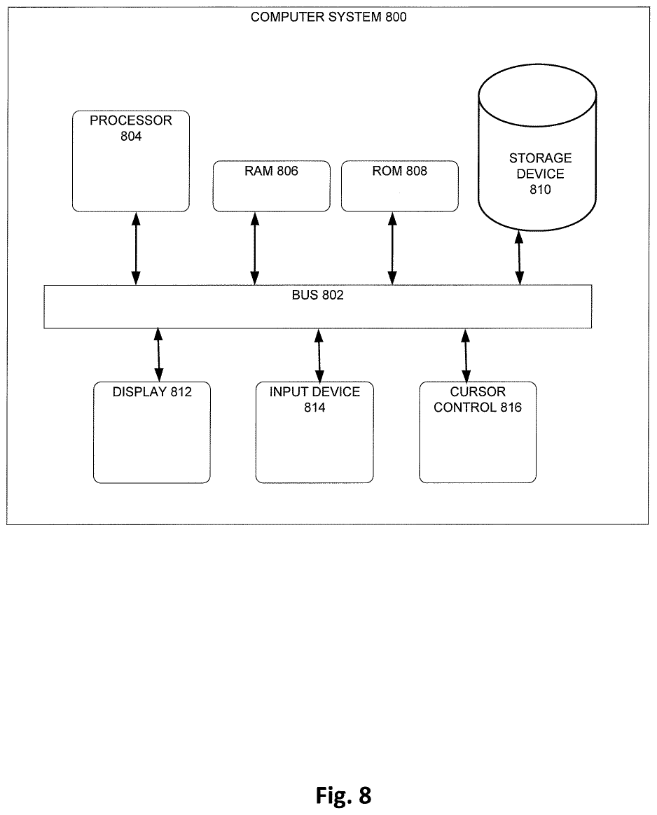

FIG. 8 is a block diagram of a computer system in accordance with various embodiments. Computer system 800 may be an example of one implementation for computing platform 102 described above in FIG. 1. In one or more examples, computer system 800 can include a bus 802 or other communication mechanism for communicating information, and a processor 804 coupled with bus 802 for processing information. In various embodiments, computer system 800 can also include a memory, which can be a random access memory (RAM) 806 or other dynamic storage device, coupled to bus 802 for determining instructions to be executed by processor 804. Memory also can be used for storing temporary variables or other intermediate information during execution of instructions to be executed by processor 804. In various embodiments, computer system 800 can further include a read only memory (ROM) 808 or other static storage device coupled to bus 802 for storing static information and instructions for processor 804. A storage device 810, such as a magnetic disk or optical disk, can be provided and coupled to bus 802 for storing information and instructions.

In various embodiments, computer system 800 can be coupled via bus 802 to a display 812, such as a cathode ray tube (CRT) or liquid crystal display (LCD), for displaying information to a computer user. An input device 814, including alphanumeric and other keys, can be coupled to bus 802 for communicating information and command selections to processor 804. Another type of user input device is a cursor control 816, such as a mouse, a joystick, a trackball, a gesture input device, a gaze-based input device, or cursor direction keys for communicating direction information and command selections to processor 804 and for controlling cursor movement on display 812. This input device 814 typically has two degrees of freedom in two axes, a first axis (e.g., x) and a second axis (e.g., y), that allows the device to specify positions in a plane. However, it should be understood that input devices 814 allowing for three-dimensional (e.g., x, y, and z) cursor movement are also contemplated herein.

Consistent with certain implementations of the present teachings, results can be provided by computer system 800 in response to processor 804 executing one or more sequences of one or more instructions contained in RAM 806. Such instructions can be read into RAM 806 from another computer-readable medium or computer-readable storage medium, such as storage device 810. Execution of the sequences of instructions contained in RAM 806 can cause processor 804 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 804 for execution. Such a medium can take many forms, including but not limited to, non-volatile media, volatile media, and transmission media. Examples of non-volatile media can include, but are not limited to, optical, solid state, magnetic disks, such as storage device 810. Examples of volatile media can include, but are not limited to, RAM 806 (e.g., dynamic RAM (DRAM) and/or static RAM (SRAM)). Examples of transmission media can include, but are not limited to, coaxial cables, copper wire, and fiber optics, including the wires that comprise bus 802.

Additionally, a computer-readable medium may take various forms such as, for example, but not limited to, a floppy disk, a flexible disk, hard disk, magnetic tape, or any other magnetic medium, a CD-ROM, any other optical medium, punch cards, paper tape, any other physical medium with patterns of holes, RAM, PROM, EPROM, EEPROM, FLASH-EPROM, solid-state memory, one or more storage arrays (e.g., flash arrays connected over a storage area network), network attached storage, any other memory chip or cartridge, or any other tangible medium from which a computer can read.

In addition to computer readable medium, instructions or data can be provided as signals on transmission media included in a communications apparatus or system to provide sequences of one or more instructions to processor 804 of computer system 800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 800 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 800, whereby processor 804 would execute the analyses and determinations provided by these engines, subject to instructions provided by any one of, or a combination of, the memory components RAM 806, ROM, 808, or storage device 810 and user input provided via input device 814.

### VI. Exemplary Descriptions of Terms

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. Section divisions (e.g., heading and/or subheadings) in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

As the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements.

As used herein, “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. “Subject” and “patient” are used interchangeably herein with respect to various embodiments.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, a “model” may include one or more algorithms, one or more mathematical techniques, one or more machine learning algorithms, or a combination thereof.

As used herein, “machine learning” may be the practice of using algorithms to parse data, learn from it, and then make one or more determinations or predictions about something in the world. Machine learning uses algorithms that can learn from data without relying on rules-based programming. A machine learning model may be a model that uses such algorithms to process input data and generate an output. Machine learning may be part of artificial intelligence.

As used herein, a “pan-indication model” may be a machine learning model capable of providing a prediction or indication of a clinical outcome independent of tumor type. In various embodiments, the pan-indication model is also capable of providing this indication independent of treatment type. The pan-indication model may include a gradient boosting model and therefore may also be referred to as a pan-indication gradient boosting model. In various embodiments, the pan-indication model includes a gradient boosting decision tree-based ensemble machine learning algorithm. In various embodiments, the pan-indication model is implemented using Extreme Gradient Boosting (XGBoost) and may be referred to as a pan-indication XGBoost model.

As used herein, a “clinical outcome’ may be a measurable change in health, function, or quality of life that results from an action. This action may be, for example, a one-time event, a periodic event, or an ongoing event. For example, the action may be a chemotherapy treatment, a therapeutic or drug treatment, or some other type of treatment or treatment protocol.

As used herein, a “treatment protocol” may describe the strategy associated with a particular type of treatment. Examples of treatments include, but are not limited to, surgery, chemotherapy, radiation therapy, stem cell or bone marrow transplantation, immunotherapy, hormone therapy, and targeted drug (e.g., therapeutic) therapy. A treatment protocol may include, for example, the plan or methodology associated with administering the particular type of treatment to a subject. For example, this plan or methodology may include a schedule for the administration of the treatment (e.g., an interval between doses), a dosing or administration amount, and/or other relevant factors.

As used herein, “longitudinal data” may include data over or corresponding to a period of time. The period of time may be in days, weeks, months, years, or some other measure of time.

As used herein, “baseline data” may include data collected at a point in time or over a period of time prior to treatment (or pre-treatment).

As used herein, a “covariate” may be a predictor or explanatory variable. For example, a covariate may be a feature associated with a subject that is used to predict one or more outcomes. In the machine learning context, a set of covariates may be a set of independent variables (or features) that can help to explain or predict the outcome/dependent variable.

As used herein, “overall survival” with respect to a particular cause (e.g., tumor growth) may mean that the subject remains alive and does not die from that cause. When data for overall survival is being used to train a machine learning model, the data may include censored data in which the recorded overall survival for a subject is the time the subject is known to have survived. For example, when definitive data regarding the length of time a subject was or is alive post-diagnosis or post-treatment is unattainable, the date for that subject's last follow-up or another data at which the subject was known to be alive may be used.

As used herein, a “hazard ratio” (or Hazard Ratio or HR) may be a measure of how often a particular event happens in one group (e.g., a treatment group) compared to how often it happens in another group (e.g., a control group), over time. For example, in tumor or cancer research, hazard ratios may be used in clinical trials to measure survival at any point in time in a group of patients who have been given a specific treatment compared to a control group given another treatment or a placebo. A hazard ratio can be used to provide an indication of overall survival. For example, a hazard ratio of one means that there is no difference in survival between the two groups. A hazard ratio of greater than one or less than one means that survival was better in one of the groups.

As used herein, a “score” may include a number, a probability, a metric, indicator, plot, graphic, notification, output, another type of output, or a combination thereof.

As used herein, “form”, “forms” or “forming” may refer to creating, generating, processing, analyzing, modifying, extracting, accessing, identifying, providing, producing, constructing, or a combination thereof.

### VII. Recitation of Example Embodiments

Embodiment 1: A method for managing progression of a clinical trial, the method comprising: forming input data for a machine learning model based on longitudinal data for a cohort of the clinical trial, wherein the input data corresponds to a plurality of input features and wherein the cohort includes a plurality of subjects; generating, for each subject in the plurality of subjects, a clinical outcome output using the machine learning model and a portion of the input data corresponding to each subject; generating a plurality of feature importance values based on the machine learning model generating the clinical outcome output for each subject in the cohort, wherein the plurality of feature importance values comprises, for each subject in the plurality of subjects, a set of feature importance values for a set of selected features from the plurality of input features; computing a ratio of interest using the plurality of feature importance values; generating an output using the ratio of interest in which the output indicates whether to allow the cohort to proceed to a next phase of the clinical trial.

Embodiment 2: The method of Embodiment 1, further comprising administering a treatment protocol to the cohort in accordance with the next phase of the clinical trial when the output includes a recommendation to proceed to the next phase of the clinical trial.

Embodiment 3: The method of Embodiment 2, wherein the treatment comprises at least one of an immunotherapy, a targeted therapy, a radiation therapy, or a chemotherapy.

Embodiment 4: The method of any one of Embodiments 1-3, wherein generating the output comprises: generating the output such that the output includes a positive recommendation that recommends proceeding to the next phase of the clinical trial when the ratio of interest is below the decision threshold; and generating the output such that the output includes a negative recommendation that recommends not proceeding to the next phase of the clinical trial when the ratio of interest is above the decision threshold.

Embodiment 5: The method of Embodiment 4, further comprising: building an operating characteristic curve for a probability of a true positive recommendation versus a false positive recommendation using historical clinical trial data; and identifying the decision threshold based on a selected risk tolerance and the operating characteristic curve.

Embodiment 6: The method of any one of Embodiments 1-5, wherein the plurality of input features includes at least one tumor growth inhibition feature.

Embodiment 7: The method of Embodiment 6, wherein the input data excludes a value for the at least one tumor growth inhibition feature for at least a portion of the plurality of subjects in the clinical trial.

Embodiment 8: The method of Embodiment 6 or Embodiment 7, wherein the at least one tumor growth inhibition feature includes at least one of tumor growth rate (KG), tumor shrinkage rate (KS), or time for tumor growth (TTG).

Embodiment 9: The method of any one of Embodiments 1-8, wherein the ratio of interest is an exponential average ratio for the plurality of feature importance values.

Embodiment 10: The method of any one of Embodiments 1-9, wherein the plurality of feature importance values includes Shapley additive explanations (SHAP) values and wherein the ratio of interest is an exponential average ratio for the SHAP values.

Embodiment 11: The method of any one of Embodiments 1-10, wherein the longitudinal data includes data corresponding to tumor size.

Embodiment 12: A method for managing progression of a clinical trial, the method comprising: generating, for each subject in a plurality of subjects in a cohort of a clinical trial, a clinical outcome output using a machine learning model and input data corresponding to a plurality of input features; generating a plurality of feature importance values based on the machine learning model generating the clinical outcome output for each subject in the plurality of subjects, wherein the plurality of feature importance values comprises, for each subject in the plurality of subjects, a set of feature importance values for a set of selected features from the plurality of input features; computing a ratio of interest using the plurality of feature importance values; generating an output using the ratio of interest, wherein the output includes a positive recommendation to allow the cohort to proceed to a next phase of the clinical trial when the ratio of interest is below a decision threshold; and administering a treatment protocol to the cohort according to a clinical trial protocol associated with the next phase of the clinical trial based on the positive recommendation.

Embodiment 13: The method of Embodiment 12, wherein the output includes a negative recommendation to not allow the cohort to proceed to the next phase of the clinical trial.

Embodiment 14: The method of Embodiment 13, further comprising: identifying a plurality of subsets of the plurality of subjects of the cohort in which each subset of the plurality of subsets has at least one differing population characteristic; repeating the step of computing the ratio of interest using the plurality of feature importance values for each subset of the plurality of subsets to form a plurality of ratios of interest; and administering a different treatment protocol to at least a portion of the cohort based on the plurality of ratios of interest.

Embodiment 15: The method of Embodiment 14, wherein the plurality of input features includes at least one of tumor growth rate (KG), tumor shrinkage rate (KS), or time for tumor growth (TTG).

Embodiment 16: A system comprising: one or more data processors; and a non-one or more data processors, cause the one or more data processors to: form input data for a machine learning model based on longitudinal data for a cohort of the clinical trial, wherein the input data corresponds to a plurality of input features and wherein the cohort includes a plurality of subjects: generate, for each subject in the plurality of subjects, a clinical outcome output using the machine learning model and a portion of the input data corresponding to each subject: generate a plurality of feature importance values based on the machine learning model generating the clinical outcome output for each subject in the plurality of subjects, wherein the plurality of feature importance values comprises, for each subject in the plurality of subjects, a set of feature importance values for a set of selected features from the plurality of input features; compute a ratio of interest using the plurality of feature importance values; generate an output using the ratio of interest in which the output indicates whether to allow the cohort to proceed to a next phase of the clinical trial.

Embodiment 17: The system of Embodiment 16, wherein generation of the output comprises: generate the output such that the output includes a positive recommendation that recommends proceeding to the next phase of the clinical trial when the ratio of interest is below the decision threshold; and generate the output such that the output includes a negative recommendation that recommends not proceeding to the next phase of the clinical trial when the ratio of interest is above the decision threshold.

Embodiment 18: The system of Embodiment 17, further comprising: build an operating characteristic curve for a probability of a true positive recommendation versus a false positive recommendation using historical clinical trial data; and identify the decision threshold based on a selected risk tolerance and the operating characteristic curve.

Embodiment 19: The system of any one of Embodiments 16-18, wherein the plurality of input features includes at least one tumor growth inhibition feature and wherein the input data excludes a value for the at least one tumor growth inhibition feature for at least a portion of the plurality of subjects in the clinical trial.

Embodiment 20: The system of any one of Embodiments 16-19, wherein the plurality of feature importance values includes Shapley additive explanations (SHAP) values and wherein the ratio of interest is an exponential average ratio for the SHAP values.

Embodiment 21: A system comprising: one or more data processors; and a non-one or more data processors, cause the one or more data processors to perform part or all of one or more methods disclosed in Embodiments 1-15.

Embodiment 22: A computer-program product tangibly embodied in a non-transitory machine-readable storage medium, including instructions configured to cause one or more data processors to perform part or all of one or more methods disclosed in Embodiments 1-15.

### VIII. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications, alternatives, and equivalents are possible within the scope of the invention claimed. Thus, it should be understood that although the present invention as claimed has been specifically disclosed by embodiments and optional features, modifications, variations, and/or equivalents of the concepts herein disclosed may be resorted to by those skilled in the art, and that such modifications, variations, and/or equivalents are considered to be within the scope of this invention as defined by the appended claims.

The description provides preferred exemplary embodiments only, and is not intended to limit the scope, applicability, or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims. For example, in describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Specific details may be provided to provide a thorough understanding of the embodiments. However, it will be understood that the embodiments may be practiced without these specific details. For example, circuits, systems, networks, processes, or other components may be shown as components in block diagram form in order not to obscure the embodiments in unnecessary detail. In other instances, well-known circuits, processes, algorithms, structures, or techniques may be shown without unnecessary detail in order to avoid obscuring the embodiments.

1. A method for managing progression of a clinical trial, the method comprising:
forming input data for a machine learning model based on longitudinal data for a cohort of the clinical trial, wherein the input data corresponds to a plurality of input features and wherein the cohort includes a plurality of subjects;
generating, for each subject in the plurality of subjects, a clinical outcome output using the machine learning model and a portion of the input data corresponding to each subject;
generating a plurality of feature importance values based on the machine learning model generating the clinical outcome output for each subject in the cohort,
wherein the plurality of feature importance values comprises, for each subject in the plurality of subjects, a set of feature importance values for a set of selected features from the plurality of input features;
computing a ratio of interest using the plurality of feature importance values; and
generating an output using the ratio of interest in which the output indicates whether to allow the cohort to proceed to a next phase of the clinical trial.

2. The method of claim 1, further comprising:
administering a treatment protocol to the cohort in accordance with the next phase of the clinical trial when the output includes a recommendation to proceed to the next phase of the clinical trial.

3. The method of claim 2, wherein the treatment comprises at least one of an immunotherapy, a targeted therapy, a radiation therapy, or a chemotherapy.

4. The method of claim 1, wherein
generating the output comprises:
generating the output such that the output includes a positive recommendation that recommends proceeding to the next phase of the clinical trial when the ratio of interest is below a decision threshold; and
generating the output such that the output includes a negative recommendation that recommends not proceeding to the next phase of the clinical trial when the ratio of interest is above the decision threshold.

5. The method of claim 4, further comprising:
building an operating characteristic curve for a probability of a true positive recommendation versus a false positive recommendation using historical clinical trial data; and
identifying the decision threshold based on a selected risk tolerance and the operating characteristic curve.

6. The method of claim 1, wherein the plurality of input features includes at least one tumor growth inhibition feature.

7. The method of claim 6, wherein the input data excludes a value for the at least one tumor growth inhibition feature for at least a portion of the plurality of subjects in the clinical trial.

8. The method of claim 6, wherein the at least one tumor growth inhibition feature includes at least one of tumor growth rate (KG), tumor shrinkage rate (KS), or time for tumor growth (TTG).

9. The method of claim 1, wherein the ratio of interest is an exponential average ratio for the plurality of feature importance values.

10. The method of claim 1, wherein the plurality of feature importance values includes Shapley additive explanations (SHAP) values and wherein the ratio of interest is an exponential average ratio for the SHAP values.

11. The method of claim 1, wherein the longitudinal data includes data corresponding to tumor size.

12. A method for managing progression of a clinical trial, the method comprising:
generating, for each subject in a plurality of subjects in a cohort of a clinical trial, a clinical outcome output using a machine learning model and input data corresponding to a plurality of input features;
generating a plurality of feature importance values based on the machine learning model generating the clinical outcome output for each subject in the plurality of subjects,
wherein the plurality of feature importance values comprises, for each subject in the plurality of subjects, a set of feature importance values for a set of selected features from the plurality of input features;
computing a ratio of interest using the plurality of feature importance values;
generating an output using the ratio of interest, wherein the output includes a positive recommendation to allow the cohort to proceed to a next phase of the clinical trial when the ratio of interest is below a decision threshold; and
administering a treatment protocol to the cohort according to a clinical trial protocol associated with the next phase of the clinical trial based on the positive recommendation.

13. The method of claim 12, wherein the output includes a negative recommendation to not allow the cohort to proceed to the next phase of the clinical trial.

14. The method of claim 13, further comprising:
identifying a plurality of subsets of the plurality of subjects of the cohort in which each subset of the plurality of subsets has at least one differing population characteristic;
repeating the step of computing the ratio of interest using the plurality of feature importance values for each subset of the plurality of subsets to form a plurality of ratios of interest; and
administering a different treatment protocol to at least a portion of the cohort based on the plurality of ratios of interest.

15. The method of claim 14, wherein the plurality of input features includes at least one of tumor growth rate (KG), tumor shrinkage rate (KS), or time for tumor growth (TTG).

16. A system comprising:
one or more data processors; and
a non-transitory computer readable storage medium containing instructions which, when executed on the one or more data processors, cause the one or more data processors to:
form input data for a machine learning model based on longitudinal data for a cohort of the clinical trial, wherein the input data corresponds to a plurality of input features and wherein the cohort includes a plurality of subjects;
generate, for each subject in the plurality of subjects, a clinical outcome output using the machine learning model and a portion of the input data corresponding to each subject;
generate a plurality of feature importance values based on the machine learning model generating the clinical outcome output for each subject in the plurality of subjects, wherein the plurality of feature importance values comprises, for each subject in the plurality of subjects, a set of feature importance values for a set of selected features from the plurality of input features;
compute a ratio of interest using the plurality of feature importance values;
generate an output using the ratio of interest in which the output indicates whether to allow the cohort to proceed to a next phase of the clinical trial.

17. The system of claim 16, wherein generation of the output comprises:
generate the output such that the output includes a positive recommendation that recommends proceeding to the next phase of the clinical trial when the ratio of interest is below a decision threshold; and
generate the output such that the output includes a negative recommendation that recommends not proceeding to the next phase of the clinical trial when the ratio of interest is above the decision threshold.

18. The system of claim 17, further comprising:
build an operating characteristic curve for a probability of a true positive recommendation versus a false positive recommendation using historical clinical trial data; and
identify the decision threshold based on a selected risk tolerance and the operating characteristic curve.

19. The system of claim 16, wherein the plurality of input features includes at least one tumor growth inhibition feature and wherein the input data excludes a value for the at least one tumor growth inhibition feature for at least a portion of the plurality of subjects in the clinical trial.

20. The system of claim 16, wherein the plurality of feature importance values includes Shapley additive explanations (SHAP) values and wherein the ratio of interest is an exponential average ratio for the SHAP values.

21. (canceled)

22. (canceled)
